(19) United States
(12) Patent Application Publication
(10) Pub. No.: US 2018/0066226 A1
(43) Pub. Date: Mar. 8, 2018

(54) CULTURE SUPPORT HAVING IMPROVED CELL ADHESIVENESS AND MOBILITY

(71) Applicant: AMOLIFESCIENCE CO., LTD., Seoul (KR)

(72) Inventors: In Yong SEO, Seoul (KR); Seung Hoon LEE, Paju-si (KR); Song Hee KOO, Seoul (KR); Ji Hyun LEE, Incheon (KR)

(21) Appl. No.: 15/560,009

(22) PCT Filed: May 10, 2016
(86) PCT No.: PCT/KR2016/004853

§ 371 Date: Sep. 20, 2017

(30) Foreign Application Priority Data
May 11, 2015 (KR) 10-2015-0065480

(51) Int. Cl. C12N 5/00

(52) U.S. Cl. CPC C12N 5/0068; C12N 2539/00; C12N 2537/00; C12N 2533/30

(57) ABSTRACT

Provided is a cell culture support having improved cell adhesion and mobility, which includes: a fibrous web which is made by accumulating fibers containing a hydrophilic polymer and a hydrophobic polymer obtained by electrospinning, in which a plurality of pores into which a culture solution is penetrated are formed.

### TECHNICAL FIELD

The present invention relates to a cell culture support, and more particularly, to a cell culture support, which can improve cell adhesion and mobility, maximize a cell survival rate by providing a culture environment familiar and suitable for cell culture, and grow cells in a desired shape and skeleton.

### BACKGROUND ART

Recently, as the use of cultured cells for the treatment of diseases has expanded, interest and research on cell culture have been increasing.

Cell culture is a technique to collect cells from living organisms and cultivate the cells in vitro. Cultured cells are used to treat diseases by differentiating them into various tissues of the body such as skin, organs, and nerves.

Such cell culture requires a culture support to provide a culture environment similar to the body.

Cells cultured on the culture support grow in an adhering state, and improving adhesion of the cells to the culture support can increase the survival rate of the cells.

Therefore, research and development of new culture supports to improve adhesion of cells and to further optimize the culture environment of cells are continuously being carried out.

Korean Patent Laid-open Publication No. 2007-0053443 discloses a method of producing a support made of a sponge-shaped fiber having a three-dimensional structure by performing a process of electrospinning a fiber spinning undiluted solution, but fibers of the support have a thread shape of a predetermined diameter and pores of the support are defined as spaces existing between the fibers.

Therefore, it is difficult for the cells to penetrate into the support via the fine pores of the support, and to grow. Thus, only the two-dimensional growth of the cells may be achieved. Therefore, there is a limit to the growth of the cells with a desired shape and skeleton. In addition, cells are attached to the support to grow, and thus there is a drawback that it is difficult to separate the cells from the support for differentiation after cell growth. This support is made of silk protein fibers, which limits the ability to enhance both the cell adhesion and the cell mobility.

### DISCLOSURE

### Technical Problem

The present invention has been made in view of the above-mentioned defects, and an object of the present invention is to provide a cell culture support which can improve cell adhesion and mobility and provide a familiar and suitable environment for cell growth.

Another object of the present invention is to provide a cell culture support capable of enabling cells to penetrate into a fibrous web to then grow, thereby enabling the cells to grow without distorting the shapes and skeletons of the cells.

### Technical Solution

In order to achieve the above object, according to an embodiment of the present invention, there is provided a cell culture support having improved cell adhesion and mobility, the cell culture support comprising: a fibrous web which is made by accumulating fibers containing a hydrophilic polymer and a hydrophobic polymer obtained by electrospinning, in which a plurality of pores into which a culture solution is penetrated are formed.

In the cell culture support having improved cell adhesion and mobility according to an embodiment of the present invention, the fibers may contain 60 wt % to 90 wt % of the hydrophilic polymer.

In the cell culture support having improved cell adhesion and mobility according to an embodiment of the present invention, the hydrophilic polymer may be PVP (polyvinylpyrrolidone) or PAN (polyacrylonitrile).

In the cell culture support having improved cell adhesion and mobility according to an embodiment of the present invention, the hydrophobic polymer may be one of PVdF (polyvinylidene fluoride), PU (polyurethane), and PES (polyethersulfone).

In the cell culture support having improved cell adhesion and mobility according to an embodiment of the present invention, the diameters of the fibers may range from 100 nm to 10 μm.

In the cell culture support having improved cell adhesion and mobility according to an embodiment of the present invention, the cell culture support may further include a plurality of beads formed on the fibers in order to secure spaces in which the cells penetrate into the fibrous web and grow therein.

In the cell culture support having improved cell adhesion and mobility according to an embodiment of the present invention, the fibrous web may be a web obtained by electrospinning a spinning solution in which the hydrophilic polymer, the hydrophobic polymer, and a solvent are mixed, and a viscosity of the spinning solution may range from 50 cps to 2000 cps.

In the cell culture support having improved cell adhesion and mobility according to an embodiment of the present invention, the diameters of the beads may be larger than the diameters of the fibers.

### Advantageous Effects

According to the present invention, a fibrous web having a plurality of pores in which fibers containing a hydrophilic polymer and a hydrophobic polymer are accumulated is embodied as a support for culturing cells, thereby improving cell adhesion and mobility, and absorbing beneficial components from secretion excreted from the cells and promoting cell growth.

According to the present invention, a cell survival rate can be maximized by implementing a cell culture support having improved cell adhesion and mobility with a fibrous web having a structure most similar to an extracellular matrix (ECM) of the human body, thereby providing a familiar and suitable environment for cell culture to thus maximize a cell survival rate.

According to the present invention, a plurality of beads suspended from the fibers of the fibrous web are formed to provide an enlarged space between the beads and the fibers and between the beads and the beads, so that the cultured cells can penetrate into the fibrous web to grow without distorting the shape and skeleton of the cells.

### BRIEF DESCRIPTION OF THE DRAWINGS

FIG. 1 is a schematic view showing a state where cells cultured in a culture support having improved cell adhesion and mobility according to an embodiment of the present invention are moved to one place.

FIGS. 2A and 2B are views for explaining the concept of cell migration according to an embodiment of the present invention.

FIG. 3 is a perspective view illustrating a state where beads are formed on a cell culture support having improved cell adhesion and mobility according to an embodiment of the present invention.

FIGS. 4A and 4B illustrate SAM photographs of fibrous webs with and without beads according to an embodiment of the present invention.

FIG. 5 is a view schematically showing a state in which cells growing inside a cell culture support according to an embodiment of the present invention are infiltrated.

FIG. 6 is a schematic view for explaining an electrospinning apparatus for manufacturing a cell culture support having improved cell adhesion and mobility according to an embodiment of the present invention.

FIG. 7 is a cross-sectional view of a cell culture support laminated according to an embodiment of the present invention.

### BEST MODE

Hereinafter, embodiments of the present invention will be described in detail with reference to the accompanying drawings.

The present invention is characterized in that a fibrous web having a plurality of pores in which fibers containing a hydrophilic polymer and a hydrophobic polymer are accumulated is implemented as a support for culturing cells to thereby improve cell adhesion and mobility.

In addition, according to an embodiment of the present invention, a fibrous web having a plurality of pores in which fibers containing a hydrophilic polymer and a hydrophobic polymer obtained by electrospinning are accumulated and formed is provided as a support for cell culture, and has a structural feature in that a plurality of beads are formed on the fibers of the fibrous web so as to form spaces to allow the cells to be cultured to penetrate into the fibrous web and grow.

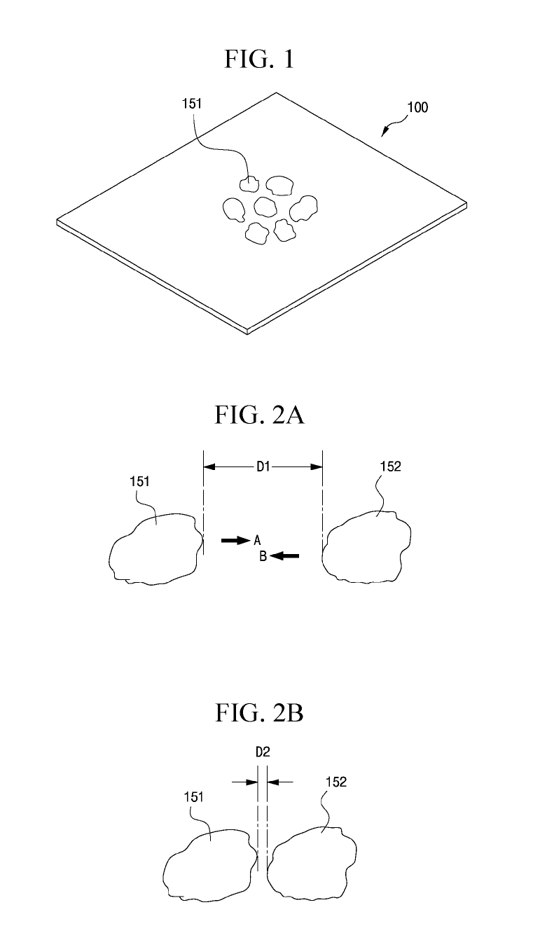

Referring to FIG. 1, a culture support having improved cell adhesion and mobility according to an embodiment of the present invention has a structure of a fibrous web 100 having a plurality of pores made by accumulating fibers.

The fibers include a hydrophilic polymer for improving cell adhesion and a hydrophobic polymer for improving cell mobility.

The fibrous web 100 is formed by accumulating fibers obtained by electrospinning a spinning solution obtained by mixing a hydrophilic polymer, a hydrophobic polymer, and a solvent, and has a plurality of pores through which a culture solution can permeate.

Cells are attached to the cell culture support of the fibrous web 100, and the cells are cultured in a state where the cells are immersed in the culture solution. Here, the hydrophilic polymer of the fibers constituting the fibrous web 100 is excellent in affinity with the culture solution, and cells attached to the cell culture support can easily absorb nutrients from the culture solution, thereby improving the growth ability, and the fibrous web 100 containing the hydrophilic polymer is excellent in hydrophilicity, so that the adhesion of cells can be enhanced.

That is, the cell culture support is immersed in the culture solution, and the cells attached to the cell culture support grow by absorbing the nutrients from the culture solution. Since cells can be attached to a support having excellent hydrophilicity, the present invention can facilitate attachment of cells by implementing a fibrous web having excellent hydrophilicity.

Here, the cells absorb the nutrients contained in the culture solution and discharge a large amount of secretion, in which the discharged secretion contains components which are beneficial for growth of the cells.

Therefore, in order to absorb the beneficial components from the secretion discharged from the cells, the cells try to move to the neighboring cells. In some embodiments of the present invention, the fibers contain a hydrophobic polymer in order to facilitate cell migration.

Accordingly, in some embodiments of the present invention, a cell culture support made of fibers containing a hydrophilic polymer and a hydrophobic polymer is implemented to improve cell adhesion and mobility.

The hydrophilic polymer may be PVP or PAN. The hydrophobic polymer may be one of PVdF, PU and PES. Alternatively, a polymer having hydrophilic or hydrophobic properties may be used in some embodiments of the present invention. However, any polymers capable of being dissolved in an organic solvent for electrospinning and capable of forming fibers by electrospinning may be used in some embodiments of the present invention, but not particularly limited thereto.

As described above, in some embodiments of the present invention, the cell culture support should have hydrophilicity and hydrophobicity. In order to enhance cell adhesion and cell mobility, it is preferable to optimize a content ratio of the hydrophilic polymer and the hydrophobic polymer in the fibers. That is, it is preferable that the fibers composed of the hydrophilic polymer and the hydrophobic polymer contain 60 wt % to 90 wt % of the hydrophilic polymer.

When the hydrophilic polymer is contained in the fibers in an amount of less than 60 wt %, the adhesion of the cells attached to the support is deteriorated. When the hydrophilic polymer is contained in the fibers in an amount exceeding 90 wt %, a small amount of the hydrophobic polymer is contained in the fibers, and thus there is a disadvantage that it is difficult for the cells to move.

As shown in FIG. 2A, when first and second cells 151 and 152 are separated from each other by a predetermined distance D1 and attached to a cell culture support to then be cultured, a large amount of secretions A and B are excreted from the first and second cells 151 and 152, respectively.

These secretions A and B include components that are beneficial for cell growth. The first and second cells 151 and 152 move as shown in FIG. 2B to absorb the beneficial components contained in the secretions A and B, and the distance D2 between the first and second cells 151 and 152 as shown in FIG. 2B becomes smaller than the distance D1 between the first and second cells 151 and 152 which are initially attached to the cell culture support as shown in FIG. 2A.

Therefore, as shown in FIG. 1, the cells 151 attached to the cell culture support 100 according to the embodiment of the present invention migrate to one region to grow, or the neighboring cells 151 become close to each other to grow.

Accordingly, in some embodiments of the present invention, a cell culture support contains a hydrophobic polymer to enhance cell mobility, thereby allowing the cells to absorb beneficial components of the secretions discharged from the cells, thus promoting cell growth.

The spinning solution for electrospinning is prepared by dissolving a hydrophilic polymer and a hydrophobic polymer in a solvent. The solvent can use at least one selected from the group consisting of DMAc (N, N-dimethyl acetoamide), DMF (N, N-dimethylformamide), NMP (N-methyl-2-pyrrolidinone), DMSO (dimethyl sulfoxide), THF (tetra-hydrofuran), EC (ethylene carbonate), DEC (diethyl carbonate), DMC (dimethyl carbonate), EMC (ethyl methyl carbonate), PC (propylene carbonate), water, acetic acid, formic acid, chloroform, dichloromethane, acetone and isopropylalcohol.

Meanwhile, since the fibrous web used as a cell culture support in some embodiments of the present invention has the structure most similar to the extracellular matrix (ECM) of the human body, the support made of the fibrous web may provide a familiar and suitable environment for cell culture to thereby maximize the survival rate of the cells.

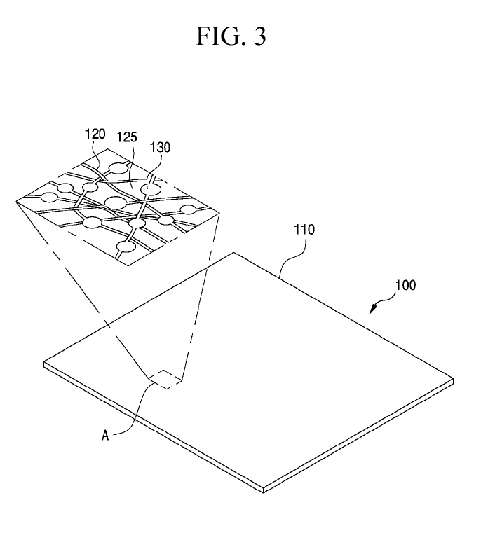

Referring to FIG. 3, a cell culture support 100 according to the embodiment of the present invention is a support to which cells are attached to culture the cells, and includes: a fibrous web 110 made by accumulating fibers 120 containing a hydrophilic polymer and a hydrophobic polymer in which a plurality of pores 125 are formed; and a plurality of beads 130 formed on the fibers 120 to secure spaces for the cells to penetrate into the fibrous web 110 and grow therein.

When referring to an enlargement view of a region ‘A’ of a fibrous web 110 in FIG. 3, fibers 120 containing a hydrophilic polymer and a hydrophobic polymer are unevenly accumulated to form a flat plate type fibrous web 110 and a plurality of pores 125 between the accumulated fibers 120.

Here, a plurality of beads 130 are formed on the fibers 120.

The diameters of the beads 130 are larger than the diameters of the fibers 120, and the beads 130 can be defined as an agglomerate of a hydrophilic polymer and a hydrophobic polymer. Here, at least one bead 130 is formed on each of all the fibers 120, or at least one bead 130 is formed on a part of all the fibers 120.

In some embodiments of the present invention, a hydrophilic polymer, a hydrophobic polymer and a solvent are mixed to prepare a spinning solution, the spinning solution is electrospun from a nozzle of a spinning apparatus to be described later to form a fiber 120 in which the bead 130 is suspended, and the fiber 120 is accumulated, to produce a fibrous web 110 for a cell culture support 100.

Here, in some embodiments of the present invention, the viscosity of the spinning solution in which a hydrophilic polymer, a hydrophobic polymer and a solvent are mixed is set to 50 cps to 2000 cps in order to realize the fibers having beads.

Here, if the viscosity of the spinning solution is less than 50 cps, the flow ability of the spinning solution is high and the droplet is sprayed from the nozzle of the spinning apparatus. If the viscosity of the spinning solution exceeds 2000 cps, the amount of an organic solvent in the spinning solution becomes small, and thus the flow ability of the spinning solution is low. In this case, only fibers are spun from the nozzle of the spinning apparatus.

The inventor(s) of the present invention confirmed through experiments that the formation of beads in the fibers 120 produced by electrospinning from the spinning nozzle is closely related to the viscosity of the spinning solution.

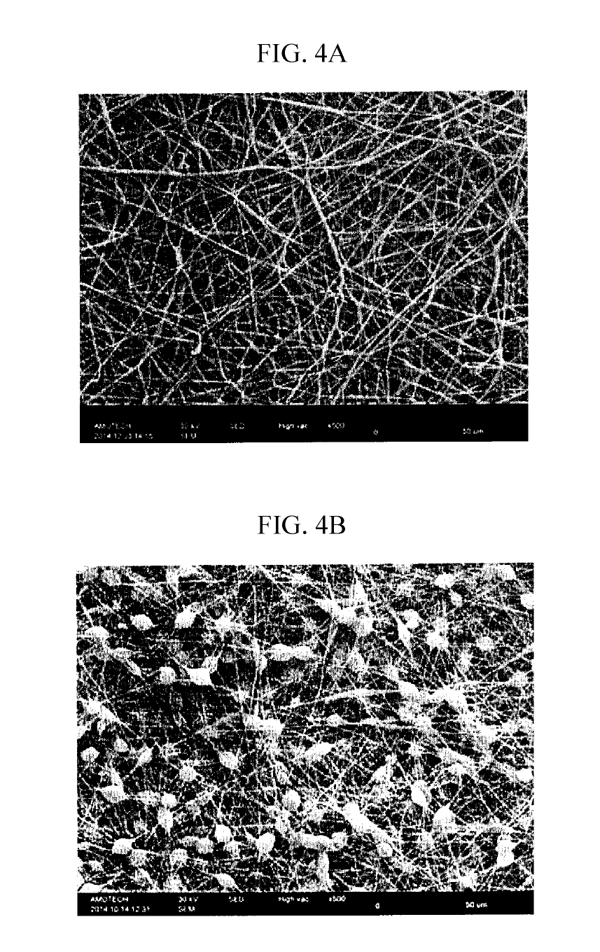

That is, a polymer was applied with a PLGA having a molecular weight of 130,000, the PLGA and a solvent were mixed so as to have a viscosity of 2100 cps and electrospun. As a result, a fibrous web consisting of only fibers was produced as shown in FIG. 4A. However, the spinning solution in which the PLGA and the solvent were mixed was electrospun so as to have a viscosity of 260 cps in order to satisfy a viscosity range set in the embodiment of the present invention, to prepare a fibrous web in which fibers having beads were accumulated as shown in FIG. 4B.

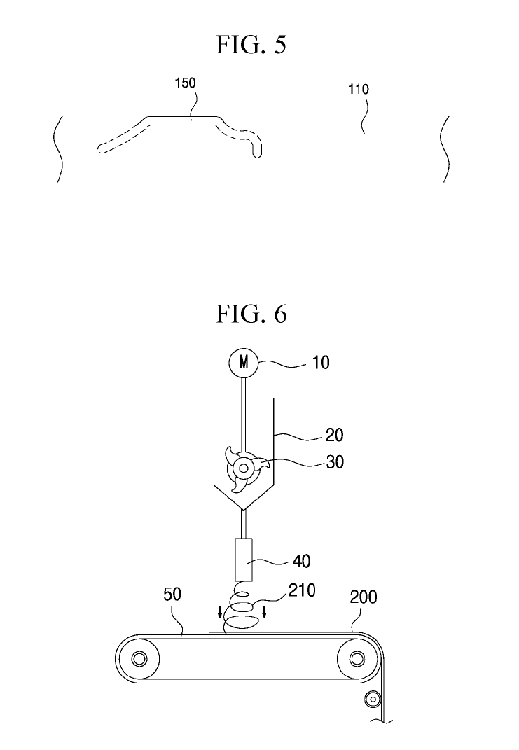

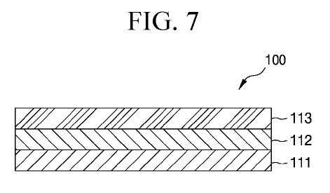

Accordingly, in some embodiments of the present invention, the cell culture support is embodied as a fibrous web containing a hydrophilic polymer and a hydrophobic polymer, and a plurality of beads suspended from the fibers of the fibrous web are provided to form enlarged spaces (i.e. large pores) between a bead and a fiber, and between one bead and another bead. Accordingly, there is an advantage that the cells 150 cultured on the fibrous web 110 can penetrate into the fibrous web 110 and grow three-dimensionally as shown in FIG. 5.

That is, the fibrous web accumulated in the fibers only forms micropores between the fibers, but the fibrous web used as the cell culture support according to some embodiments of the present invention has pores between the bead and the fiber and between the beads. Therefore, the pores of the fibrous web according to some embodiments of the present invention in which beads are present become pores larger than the micropores formed between the fibers of the fibrous web in which beads are not present, and become spaces facilitating the penetration of the cells 150 to be grown .

FIG. 6 is a schematic view illustrating an electrospinning apparatus for preparing a cell culture support according to an embodiment of the present invention.

Referring to FIG. 6, an electrospinning apparatus for producing a cell culture support according to an embodiment of the present invention is characterized in that a stirring tank 20 for supplying a stirred spinning solution is connected to a spinning nozzle 40, a grounded collector 50 in the form of a conveyor that moves at a constant speed is placed in a lower portion of the electrospinning apparatus and spaced from the spinning nozzle 40, and the spinning nozzle 40 is connected to a high voltage generator.

Here, a hydrophilic polymer, a hydrophobic polymer, and a solvent are mixed with a stirrer 30 to prepare a spinning solution. Here, a pre-mixed spinning solution may be used before being put into the electrospinning apparatus without mixing a biodegradable polymer and a solvent in the stirrer 30.

Thereafter, when a high voltage electrostatic force is applied between the collector 50 and the spinning nozzle 40, the spinning solution is spun by the spinning nozzle 40 into the ultrafine fibers 210 to then be emitted to the collector 50. The fibers 210 are accumulated to the collector 50 to form the fibrous web 200 of the cell culture support.

More specifically, the spinning solution discharged from the spinning nozzle 40 is discharged as the ultrafine fibers 210 while passing through the spinning nozzle 40 charged by the high voltage generator, and the ultrafine nanofibers 210 are sequentially laminated on the grounded collector 50 provided in the form of a conveyor moving at a certain speed to form the fibrous web 200 of the cell culture support.

While the present invention has been particularly shown and described with reference to exemplary embodiments thereof, by way of illustration and example only, it is clearly understood that the present invention is not to be construed as limiting the present invention, and various changes and modifications may be made by those skilled in the art within the protective scope of the invention without departing off the spirit of the present invention.

### INDUSTRIAL APPLICABILITY

The present invention is applicable to a cell culture support capable of maximizing a cell survival rate by improving cell adhesion and mobility and providing a culture environment familiar and suitable for cell culture, and growing cells in a desired shape and skeleton.

What is claimed is:

1. A cell culture support having improved cell adhesion and mobility, the cell culture support, which is a support to which cells are attached to culture the cells, the cell culture support comprising:
a fibrous web which is made by accumulating fibers containing a hydrophilic polymer and a hydrophobic polymer obtained by electrospinning, in which a plurality of pores into which a culture solution is penetrated are formed.

2. The cell culture support having improved cell adhesion and mobility of claim 1, wherein the fibers contain 60 wt % to 90 wt % of the hydrophilic polymer.

3. The cell culture support having improved cell adhesion and mobility of claim 1, wherein the hydrophilic polymer is PVP or PAN.

4. The cell culture support having improved cell adhesion and mobility of claim 1, wherein the hydrophobic polymer is one of PVdF, PU, and PES.

5. The cell culture support having improved cell adhesion and mobility of claim 1, wherein the diameters of the fibers range from 100 nm to 10 μm.

6. The cell culture support having improved cell adhesion and mobility of claim 1, further comprising a plurality of beads formed on the fibers in order to secure spaces in which the cells penetrate into the fibrous web and grow therein.

7. The cell culture support having improved cell adhesion and mobility of claim 6, wherein the fibrous web is a web obtained by electrospinning a spinning solution in which the hydrophilic polymer, the hydrophobic polymer, and a solvent are mixed, and a viscosity of the spinning solution ranges from 50 cps to 2000 cps.

8. The cell culture support having improved cell adhesion and mobility of claim 6, wherein the diameters of the beads may be larger than the diameters of the fibers.
